(19) United States
(12) Patent Application Publication
(10) Pub. No.: US 2009/0030495 A1
(43) Pub. Date: Jan. 29, 2009

(54) SYSTEM FOR CONTROLLED PROSTHESIS DEPLOYMENT

(75) Inventor: INA KOCH, Villingen-Schwenningen (DE)

Correspondence Address: FULWIDER PATTON, LLP (ABBOTT), 6060 CENTER DRIVE, 10TH FLOOR, LOS ANGELES, CA, 90045, US

(73) Assignee: Abbott Laboratories Vascular Enterprises Limited, Dublin (IE)

(21) Appl. No.: 11/828,297

(22) Filed: Jul. 25, 2007

(51) Int. Cl. A61F 2/84

(52) U.S. Cl. 623/1.11

(57) ABSTRACT

A system for controlled deployment of a prosthesis, in which a sheath is retracted from a prosthesis in a body vessel or duct. The system includes a handle for deploying the prosthesis, the handle comprises a base member fixed to a catheter, and a slide member for sliding past the base member, the slide member being attached to an end of a retraction member. The base member includes an opening having the general shape of a rectangular wave for interacting with the slide member, whereby, repeated manipulation of the slide member causes the sheath to be retracted.

### BACKGROUND OF THE INVENTION

The present invention relates to an implant delivery and deployment system. More specifically, the invention relates to a deployment system that uses a handle operable under automatically controlled conditions to withdraw the retractable outer sheath and deploy a medical implant for minimally invasive application, such as an endovascular stent graft, vena cava filter, self-expanding stent, balloon expandable stent, and the like.

Delivery systems for deploying a medical implant, such as an endovascular stent graft, vena cava filter, self-expanding stent, balloon expandable stent or the like, are known in the field of medical technology. These medical devices have many uses and applications. In particular, a stent is a prosthesis or implant which is generally tubular with openings formed therein, and which is expanded radially in a vessel or lumen to improve and reinforce the vessel's patency. Stents are widely used in body lumens, body canals, ducts or other body vessels. A self-expanding stent is a stent which expands from a first compressed condition during delivery to a second expanded condition when released from the delivery device, where the stent exerts outward radial force on a portion of the body vessel to improve and maintain patency. One common self-expanding stent is manufactured of Nitinol, a nickel-titanium shape memory alloy, which can be formed and annealed, compressed from its original shape and held at a low temperature, and recalled to its original shape with heating, such as when deployed at body temperature in the body.

Pull-back stent delivery systems are known in the art. However, one important need felt in the art for delivering a stent or other prosthesis/implant is the ability to pull back or retract the outer sheath from the prosthesis in a controlled and precise manner, to enable the physician to accurately determine proper positioning of the prosthesis as well as track the retraction of the outer sheath within the patient. Although devices exist in the art for retracting an outer sheath from its position surrounding a prosthesis loaded onto a catheter, these are typically complex and expensive to manufacture, and tend to have little or no safeguard to prevent accidental instantaneous deployment of the entire stent. For example, in some devices, if the user overextends a single hand movement, the sheath may retract completely from the prosthesis, with the result that the prosthesis is deployed too soon with potentially catastrophic results.

Accordingly, there is a need in the art for a system that will prevent the user from too rapidly removing a sheath from a prosthesis. There is also a need for such a system that is simple to fabricate and economical to manufacture, allowing for disposal after use. The present invention addresses these and other needs.

### SUMMARY OF THE INVENTION

In a preferred embodiment there is described a system for the controlled delivery of a prosthesis or implant in a body vessel or duct. The delivery system includes an elongate core cylinder that forms part of a catheter, and a prosthesis or implant surrounding the core cylinder. A sheath surrounds the prosthesis. A handle is provided for deploying the prosthesis from its position of confinement by the sheath wherein the handle comprises a base member operably connected to the core cylinder. The base member has an elongate axis and defines a shaped opening having the general shape of a rectangular wave. A slide member is provided, and is operably attached to the sheath. The slide member is moveable in relation to the base member, whereby, the sheath is movable in relation to the stent.

A protrusion on the slide member extends through the shaped opening, and a biasing member is provided to urge the slide member in relation to the base member. By repeatedly moving the protrusion along portions of the opening extending perpendicular to the base member's axis, this action permits the biasing member to incrementally move the slide member proximally in relation to the base member, and thereby to retract the sheath from the prosthesis. (As used herein, “proximally” refers to the end or direction closer to the user of the delivery system, “distal” refers to the end or direction of the delivery system remote from the user and closer to the patient.) Thus, the rectangular wave shape of the opening has a “meander pattern” which provides a safety mechanism for controlling the retraction of the sheath from the prosthesis, and prevents sudden catastrophic prosthesis deployment by sheath retraction. The user manipulates the protrusion laterally across the base member, while the biasing member incrementally moves the piston in relation to the base member, and thereby retracts the sheath from the prosthesis. The shape of the opening forces the retraction process to stop every few millimeters, and allows the user to angiographically check that the prosthesis is correctly positioned before making the next manipulation.

In preferred aspects of the invention, the sheath is operably attached to the slide member via a retraction member that includes a tubular portion surrounding the core cylinder. Again preferably, the base member includes a circular cylinder, and the slide member includes a piston slidable within the circular cylinder. In this preferred embodiment, the piston can rotate to some degree about the axis of the base member, the protrusion is a pin, and the biasing member is a helical spring, with an elongate axis that is coaxial with the axis of the cylinder.

In a more detailed aspect of the invention, at least one of the portions of the opening extending generally parallel to the axis of the base member includes a curved portion. The curved portion may have a single curvature, or it may have a double curvature. In another detailed aspect, at least one of the portions of the opening extending generally perpendicular to the axis of the base member includes a curved portion. The curved portions are configured to slow down the longitudinal movement of the slide member under the urging of the biasing member, to avoid damage to the system.

In another aspect, the invention includes a method of retracting a sheath to uncover a prosthesis mounted on a catheter. This aspect includes attaching the catheter to a base member having a first elongate axis, and operably attaching the sheath to a slide member having a second elongate axis, the slide member being slidable parallel to the first axis. The slide member is biased proximally in relation to the base member. Then the slide member is incrementally moved proximally in relation to the base member by rotating the slide member, clockwise and, alternatingly, counterclockwise, about the second axis. In this way, the sheath is incrementally retracted proximally to uncover the prosthesis. Preferably, incrementally moving the slide member proximally includes causing the slide member to follow a path determined by a shaped opening in the base member, and this may include manipulating a pin extending from the slide member through the shaped opening. Thus, the shaped opening provides a safety feature that prevents the biasing member from causing the sheath to retract from the prosthesis in a single catastrophic movement, but requires a series of incremental movements allowing the user to check on the retraction after increment.

The present invention is applicable to deployment of devices that may be left behind in a vessel or duct, such as a stent, and also to devices that may be removed from the vessel or duct after deployment, such as a balloon on its own, not associated with a stent. Accordingly, the terms “implant” and “prosthesis” shall be used herein as not requiring that some structure be left behind in the vessel or duct after deployment, and will cover a balloon alone, removed after deployment.

These, and other features of the invention, will be disclosed more fully in the detailed description of the preferred embodiments that follow, and the drawings attached.

### BRIEF DESCRIPTION OF THE DRAWINGS

FIG. 1 is a schematic view, in partial section, depicting a controlled prosthesis delivery system having features of the present invention.

FIG. 2 is a sectional view of handle portion of the above invention.

FIG. 3 is a sectional view taken substantially along the line 2-2 of FIG. 2

FIG. 4 is a sectional view taken substantially along the line 4-4 of FIG. 1.

FIG. 5 is a schematic view of a further embodiment of a handle portion having features of the present invention.

FIG. 6 is schematic view of yet a further embodiment of a handle portion having features of the present invention.

### DETAILED DESCRIPTION OF THE PREFERRED EMBODIMENTS

With reference to the drawings, which are provided by way of exemplification and not limitation, there is described a controlled prosthesis deployment system having features of the present invention.

A preferred embodiment of the invention includes a release handle, generally identified by the numeral 20. The release handle is adapted to operate in conjunction with a catheter 22. The catheter is configured to remotely deliver a prosthesis in a body vessel or duct.

Catheters for remotely delivering a prosthesis are known, and may include a core cylinder 27 having an internal lumen 28 (FIG. 4). A guidewire 32 may be inserted within the lumen 28, according to known usage wherein the guidewire is first threaded into a body vessel (not shown), whereafter the catheter 22 is run up into the body vessel over the guidewire 32. At the distal end of the catheter, a prosthesis may be positioned to surround the catheter. In FIG. I it is shown that the prosthesis may be a self-expanding stent 34, although the present invention may work in conjunction with other types of prostheses. Additionally, an inflatable balloon 35 may be positioned on the catheter for insertion within the stent after the stent is deployed, whereupon inflation of the balloon may position the stent at the correct diameter according to known procedures. An outer retractable sheath 36 is positioned to surround the stent 34 so that, when the catheter 22 is introduced into a body vessel, the stent is confined and protected within the sheath 36 where it will not cause trauma to the walls of the body vessel nor prematurely deploy. Subsequent retraction of the sheath 36 to uncover the stent allows the stent to expand. Once the stent is correctly positioned within the body vessel (which positioning may include balloon inflation), the catheter 22 is withdrawn, leaving the stent to reinforce the body vessel and maintain its patency.

In a preferred embodiment, a retraction member 24 may be provided for exerting a retraction force to the sheath 36. The distal end of the retraction member 24 is connected to the sheath 36 at a connection point 37, so that any proximal displacement imparted to the retraction member 24 is also imparted to the sheath 36. The proximal end of the retraction member is connected to the release handle 20 as described in more detail below. Preferably, the connector point 37 may be an annulus configured to transfer a tensile load between the cylindrical retraction member 24 and the cylindrical sheath 36.

The retraction member 24 may be a rod, wire or hypotube, that can be made of a metal or polymeric material. In the embodiment disclosed in the figures, the retracting member 24 is positioned to surround the core cylinder 27. The retraction member lumen 26 (FIG. 4) may additionally carry flushing fluid for purging and cleaning the catheter. If the retraction member 24 is a hypotube, the hypotube can be configured to carry the purging fluid. Additionally, purging fluid may be introduced into the catheter 22 generally via a standard lure connector 41 positioned at the proximal end of the catheter.

The retractable sheath 36 may be flexible or rigid, but in a preferred embodiment is flexible to facilitate delivery of the catheter 22 to remote portions of the body vessel. If an inflatable balloon 35 is provided, the sheath 36 may also cover the balloon. Radiopaque markers 39 may be positioned along the length of the catheter to mark the positions of the balloon 35 and the stent 34 in order to facilitate delivery of the stent using known methods of radiography.

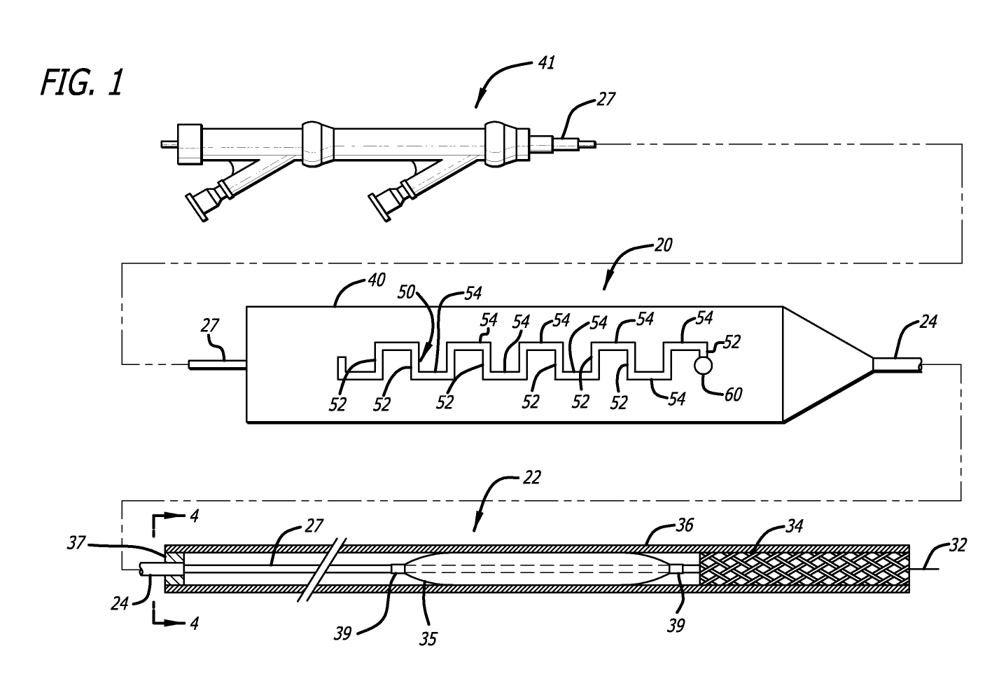

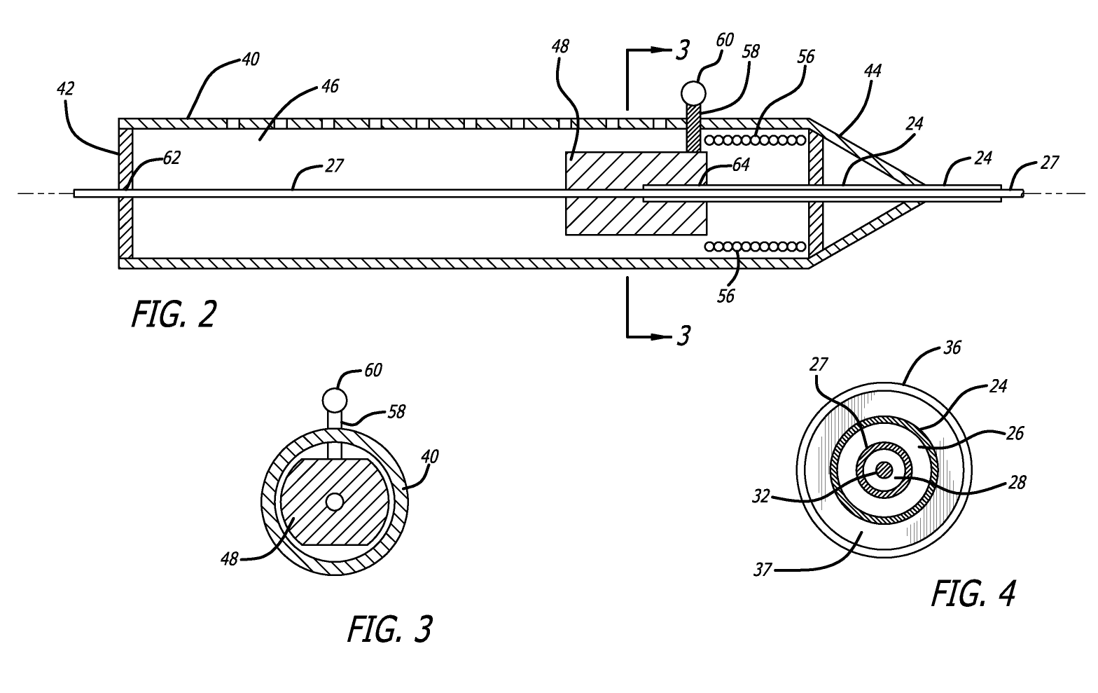

Turning now to FIGS. 1-3, there is exemplified a release handle 20 having features of the present invention for activating the catheter 22 to deploy the prosthesis 34. A base member is provided for grasping and protecting the components of the handle. In a preferred embodiment, the base member is a cylinder 40 having an elongate axis with a proximal end 42 and a distal end 44 and an internal bore 46. The cylinder 40 is provided for manually grasping the control handle 20 and for protecting the inner workings of the handle. A slide member is configured to slide along the base member. Where the base member is a cylinder, the slide member is a piston 48 (FIGS. 2-3) configured to slidingly reciprocate within the bore 46 of the cylinder 40. Preferably, the piston is configured to fit snugly within the bore of the cylinder to eliminate wobble, and may be lubricated.

A shaped opening 50 (FIG. 1) is cut into the wall of the base member, or cylinder, 40. The shaped opening has a generally wavelike configuration. Portions 52 of the opening 50 extend perpendicularly across the longitudinal axis of the cylinder, while other portions 54 extend parallel to the longitudinal axis of the cylinder, to produce an opening 50 generally having the shape of a rectangular wave.

A biasing member 56 (FIG. 2), preferably a helical spring, is positioned within the cylinder to urge the piston 48 proximally along the cylinder bore 46. In a preferred embodiment, the biasing member 56 is located distal to the piston 48, as exemplified in FIG. 2, so that, when the piston 48 is loaded by moving it toward the distal end 44 of the cylinder 40, the biasing member 56 is compressed and urges the piston 48 toward the proximal end 42 of the cylinder.

The piston 48 has a protrusion that is configured to pass through the opening 50 in the cylinder 40. In a preferred embodiment, the protrusion is a pin 58 that may be screwed into the piston. A knob 60 is fitted atop the protrusion or pin 58, to facilitate manual control of the pin and the attached piston.

Turning now to the interaction between the catheter 22 and the control handle 20, the retraction member 24 is fixed to the piston 48 at a connection point 64 (FIG. 2), while the core cylinder 27 is fixed to the cylinder at a connection point 62. Thus, it will be appreciated, retraction of the piston 48 in relation to the cylinder 40 will result in retraction of the retraction member 24 in relation to the core cylinder 27, and thus retraction of the sheath 36 in relation to the stent 34.

The system for controlled delivery of a prosthesis is assembled for use as follows. The handle 20 is first “loaded” by moving the piston 48 to a distal position in the cylinder 40, as exemplified in FIG. 2, against the urging of the biasing member 56. This action may require the pin 58 to be threaded distally up the length of the opening 50, weaving it along the shape of the opening as necessary. Once the pin is in a distal position, because the opening 50 is shaped with portions 52 extending perpendicular to the direction in which the biasing member 56 urges the piston 48 and the connected pin 58, the pin will be held longitudinally immobile in the opening 50 when it is in any one of these perpendicular portions 52. While the piston and pin are held in this stable immobile condition, preferably with the pin in the most distal perpendicular portion 52, the catheter 22 is affixed to the handle, as described above, in which the core cylinder 27 is fixed to the cylinder 40 at connection point 62, and the retraction member 24 is fixed to the piston 48 at connection point 64. Connection points 62 and 64 may be achieved by adhesive, compressive, frictional, or similar means. It will be appreciated that it is not essential to connect the sheath to the piston via a retraction member (the sheath could be directly connected to the piston), but the objective is to operably connect the sheath to the piston whereby movement of the piston in relation to the base member causes movement of the sheath in relation to the core cylinder upon which the prosthesis is mounted. Connecting the sheath to the piston via a narrower retraction member as described is a preferred embodiment of the invention in that such permits a reduced diameter over much of the length of the catheter 22.

Turning now to actual operation of the system once it is loaded, the physician first inserts the catheter 22 with mounted prosthesis 34 into a body vessel (not shown) following a standard method known in the art. When the physician is satisfied that the prosthesis is in the correct position in relation to the body vessel and wishes to retract the sheath 36 to deploy the prosthesis 34, she moves the knob 60 (with its pin 58) laterally across the first portion 52 of the opening 50 that extends perpendicularly across the cylinder axis. When the pin reaches the end of such portion, the biasing member 56 urges the piston 48 proximally so that the pin travels proximally down the length of portion 54 extending parallel to the cylinder axis until the pin 58 reaches the end of its travel along that portion 54 and reaches the next laterally extending portion 52 of the opening, whereupon the movement of the pin (and piston) is again arrested. Thereafter, after checking the position of the stent within the body vessel, the physician moves the knob 60 (with its pin 58) laterally across the cylinder in the opposite direction to the first manipulation, and the piston 48 is urged proximally by another longitudinal increment along the opening 50. Following this technique, the piston 48 can be moved proximally along the length of the handle 20 under a controlled methodology, and the sheath 36 can be completely retracted from the prosthesis 34 to permit deployment. It will be appreciated that the progress of the piston 48 proximally down the length of the cylinder 40 involves rotating the piston, alternatingly, clockwise and counterclockwise in between incremental longitudinal movements in which the biasing member 56 urges the piston axially. The path followed by the piston is determined by the shape of the opening 50, and the user may elect to terminate retraction at any time by not manipulating the pin further.

It will be appreciated that, in this way, the physician operator can control the retraction of the sheath 36 from the prosthesis 34 in short incremental stages. The structure described is extremely simple, it requires few parts, and is therefore inexpensive to manufacture so as to permit disposal of the entire unit after use. The introduction of a “meander pattern” into the cylinder of the handle means that retraction of the sheath cannot take place with catastrophic speed if the physician accidentally loses control of the button 60. This provides an effective, yet inexpensive, solution to the problem of providing a system for deployment of a prosthesis in a body vessel over which the operator cannot accidentally lose control.

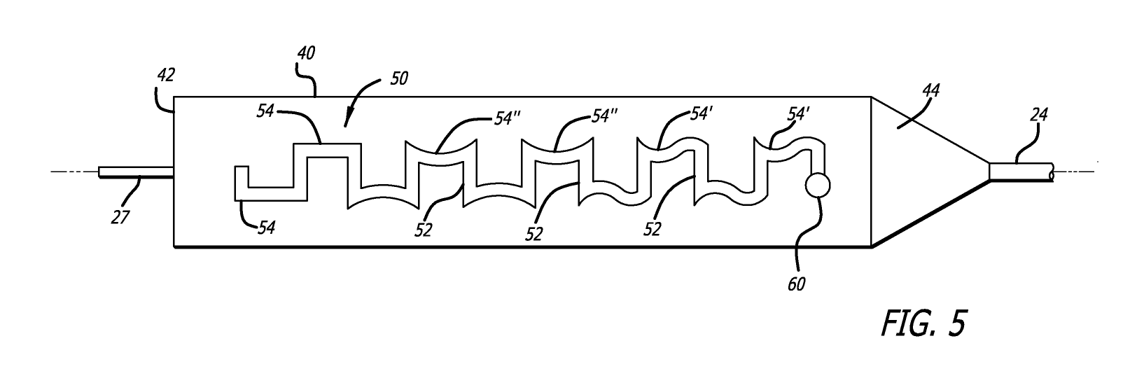

In another embodiment of the invention, exemplified in FIG. 5, the shaped curve 50 in the base member may have portions 54′, and 54″, that extend generally parallel to the axis of the base member, but that are not substantially straight. Rather, they are substantially curved. This feature may be applied to all the portions extending generally parallel to the axis, or to only some of those portions, as exemplified in FIG. 5. The advantage of this aspect is that the curved shape will slow down the rapid movement of the piston under force of biasing member 56, especially when the piston 48 is near the distal end 44 of the cylinder 40 and the biasing member 56 is compressed to its maximum extent. If the parallel portions 54 are substantially straight (as exemplified in FIG. 1), there is a possibility that the pin 58 may move too rapidly from one end to the other and impact the starting point of the next perpendicular portion 52 of the curve 50 with such force that damage may be caused. The shape of the curved parallel portion may be a single arc of a circle such as exemplified by portion 54″ (having a single curvature) or an approximately sinusoidal shape in which the direction of curvature changes from positive to negative (having a double curvature), as exemplified by portion 54′ in FIG. 5. In each case, the direction of the portion 54′, 54″, as it joins with perpendicular portion 52 preferably has a vector pointing away from the axis of the cylinder 40 when viewed from directly above the opening 50, as seen in FIG. 5. This feature tends to bring the pin 58 to a stop as it reaches portion 52. (If the vector were in the other direction, the pin might tend to slide some distance along portion 52 before coming to a stop. It will be appreciated that this would introduce an element of risk in the event the pin were to slide along the whole length of portion 52.)

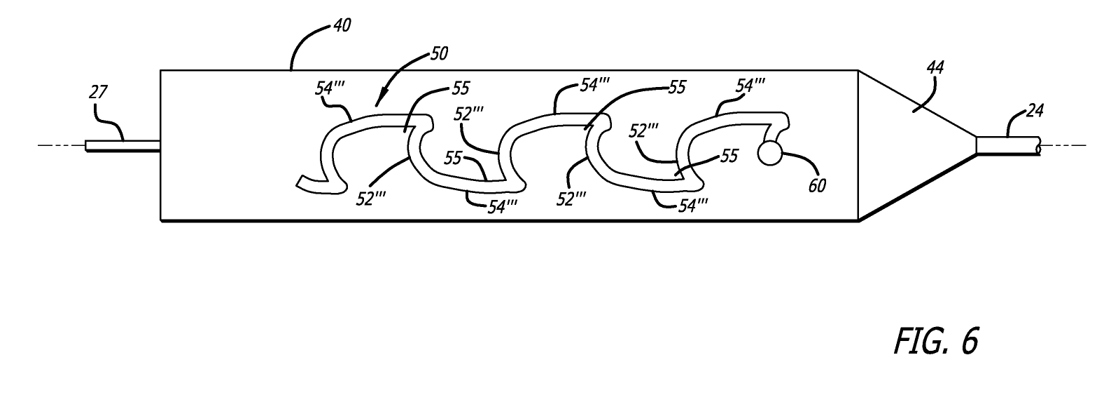

In yet a further embodiment of the invention, exemplified in FIG. 6, the generally rectangular curve 50 in the base member may be configured so that both the portions that extend parallel 54′″ and the portions that extend perpendicular 52′″ to the axis of the base member 40 are curved. The curve shape of each may be ergonomically shaped to dampen the impact of the pin when it comes to the end of its travel along one half wave length, without coming to a sudden stop that may cause damage. Thus, the shape of the perpendicular portion 52′″ may have a curvature that tends to return the pin in a distal direction before the pin comes to a stop, and may allow for the pin to initially overshoot an equilibrium position, to be returned proximally to the equilibrium position by the action of the biasing member 56. A small return bump 55 is included at the end of each perpendicular portion 52′″ to stop the pin 58 from carrying on by its own inertia into the next parallel portion 54′″. When the user wishes to advance the piston by the next increment, she may manually nudge the pin 58 over the edge of the bump 55.

Thus, there has been described a novel system and method for the controlled deployment of a prosthesis. The system involves few parts, each of which is simple and lightweight, and therefore inexpensive to manufacture, allowing disposal after use.

It will be realized that the foregoing preferred specific embodiments have been shown and described for the purpose of illustrating the functional and structural principles of this invention and are subject to change without departure from such principles. Therefore, this invention includes all modifications encompassed within the spirit and scope of the following claims.

I claim:

1. A deployment system for deploying a prosthesis in a duct, comprising:
an elongate core cylinder for delivering a prosthesis to a predetermined location in a duct;
a prosthesis surrounding the core cylinder;
a sheath surrounding the prosthesis;
a handle for deploying the prosthesis, the handle comprising:
a base member connected to the core cylinder, the base member having an elongate axis and defining a shaped opening having the general shape of a rectangular wave wherein some portions of the opening extend generally parallel to the axis of the base member, and some portions extend generally perpendicular to the axis of the base member;
a slide member operably attached to the sheath, the slide member being moveable in relation to the base member, whereby, when the slide member is moved in relation to the base member, the sheath is moved in relation to the prosthesis;
a protrusion on the slide member extending through the shaped opening;
a biasing member positioned to urge the slide member in relation to the base member;
whereby, repeated manual movement of the protrusion along those portions of the opening extending perpendicular to the base member's axis causes the biasing member to incrementally retract the sheath from the prosthesis.

2. The deployment system of claim 1, wherein the slide member is operably attached to the sheath via a retraction member.

3. The deployment system of claim 2, wherein the retraction member includes a tubular portion that surrounds the core cylinder.

4. The deployment system of claim 1, wherein the base member includes a circular cylinder.

5. The deployment system of claim 4, wherein the slide member includes a piston slidable within the circular cylinder.

6. The deployment system of claim 5, wherein the piston can rotate to some extent about the axis of the base member.

7. The deployment system of claim 1, wherein the protrusion is a pin.

8. The deployment system of claim 1, wherein the biasing member is a spring.

9. The deployment system of claim 8, wherein the spring is a helical spring.

10. The deployment system of claim 8, wherein the spring has an elongate axis that is coaxial with the axis of the base member.

11. The deployment system of claim 1, wherein at least one of the portions of the opening extending generally parallel to the axis of the base member includes a curved portion.

12. The deployment system of claim 11, wherein the curved portion has a single curvature.

13. The deployment system of claim 11 wherein the curved portion has a double curvature.

14. The deployment system of claim 1, wherein at least one of the portions of the opening extending generally perpendicular to the axis of the base member includes a curved portion.

15. The deployment system of claim 14, wherein the curved portion has a bump configured to momentarily direct the protrusion in a distal direction as the protrusion advances generally in a proximal direction along the opening under the urging of the biasing member.

16. A method of retracting a sheath to uncover a prosthesis mounted on a catheter, comprising:
attaching the catheter to a base member having a first elongate axis, the base member being positioned proximal to the catheter;
operably attaching the sheath to a slide member having a second elongate axis, the slide member being slidable parallel to the first axis, biasing the slide member proximally in relation to the base member;
incrementally moving the slide member proximally in relation to the base member by rotating the slide member about the second axis, whereby the sheath is incrementally retracted proximally to uncover the prosthesis.

17. The method of claim 16, wherein rotating the slide member about the second axis includes rotating the base member clockwise and, alternatingly, counterclockwise.

18. The method of claim 16, wherein rotating the slide member about the second axis includes manually rotating the slide member.

19. The method of claim 16, wherein incrementally moving the slide member proximally includes causing the slide member to follow a path determined by a shaped opening in the base member.

20. The method of claim 19, wherein causing the slide member to follow a path determined by a shaped opening in the base member includes manipulating a protrusion extending from the slide member through the shaped opening.

21. The method of claim 16, wherein incrementally moving the slide member proximally includes moving the slide member proximally under the bias of a spring.
